(19) United States
(12) Patent Application Publication
(10) Pub. No.: US 2011/0029029 A1
(43) Pub. Date: Feb. 3, 2011

(54) IMPLANTABLE PACEMAKER DEVICE THAT USES IMPEDANCE CARDIOGRAPHY

(76) Inventors: James A. Leibsohn, River Hills, WI (US); Anna Leibsohn, River Hills, WI (US)

Correspondence Address: QUARLES & BRADY LLP, 411 E. WISCONSIN AVENUE, SUITE 2040, MILWAUKEE, WI, 53202-4497, US

(21) Appl. No.: 11/912,191

(22) PCT Filed: Apr. 19, 2006
(86) PCT No.: PCT/US06/14730

§ 371 Date: Oct. 21, 2010

Related U.S. Application Data
(60) Provisional application No. 60/673,686, filed on Apr. 21, 2005.

(51) Int. Cl. A61N 1/368; A61N 1/02

(52) U.S. Cl. 607/3; 607/24

(57) ABSTRACT

An implantable pacemaker that uses impedance cardiography to measure intrathoracic impedance and then transmit impedance data to an external PC based analyzer for accurate calculation of cardiac output, and a method for optimizing cardiac resynchronization therapy using the pacemaker are disclosed.

### CROSS-REFERENCES TO RELATED APPLICATIONS

This application claims priority from U.S. Provisional Patent Application No. 60/673,686 filed Apr. 21, 2006.

### STATEMENT REGARDING FEDERALLY SPONSORED RESEARCH

Not Applicable.

### BACKGROUND OF THE INVENTION

1. Field of the Invention

The invention relates to a Cardiac Resynchronization Therapy (CRT) implantable pacemaker that uses impedance cardiography to calculate cardiac output, and to a method for optimizing cardiac resynchronization therapy using the pacemaker.

2. Description of the Related Art

Congestive heart failure and left ventricular dysfunction are the result of multiple and common disease processes, including coronary disease, hypertension, diabetes, cardiomyopathy to name but a few. It will be responsible for more deaths than all other causes combined by 2010, and accounts now for the lion's share of health care cost in the United States.

Current therapies available today include surgery, such as bypass or valve surgery, or more recently, ventricular remodeling surgery; medical therapy including medications known as ACE inhibitors, beta blockers, aldosterone antagonists, and so forth; and recently, the application of cardiac resynchronization therapy in selected patients. Cardiac resynchronization therapy, CRT, as it is known, is a pacemaker based technology in which leads are placed in both left and right ventricles in order to synchronize their contraction and thus optimize cardiac function. Cardiac resynchronization therapy systems have been developed by various medical device companies.

Approximately one fourth of patients receiving cardiac resynchronization therapy fail to respond favorably to this very expensive and labor intensive therapy, and in such patients, attempts are made to optimize the many adjustable parameters of the cardiac resynchronization therapy device. Optimization is conducted by confirming proper position of the leads, but primarily by Doppler analysis of blood flow characteristics during echocardiographic evaluation, a moderately expensive procedure which takes about 30 to 45 minutes to perform, and is occasionally technically inadequate for purpose of optimization.

Impedance cardiography is a technique by which there is made an indirect measurement of the cardiac output, or volume of blood pumped by the heart in the time of one minute, wherein the cardiac output is calculated from the measured intrathoracic impedance. To this point in time, impedance cardiography is performed much like an electrocardiogram, that is, with wires placed on the skin of the chest and extremities. A tiny current is introduced between two points (a current source and a current sensor) placed some distance from one another on the chest, the impedance of the current is analyzed and is translated into the cardiac output.

Impedance cardiography is an older science and has historically been slow to evolve due to the fact that the measurement of impedance is affected by many variables, typically resulting in spurious results and therefore unsupported assumptions. Proprietary algorithms have been developed to increase the accuracy of the calculation of cardiac output from impedance measurements, and it is felt that these algorithms have solved issues addressing accuracy of cardiac output calculation. In the future, concepts of impedance cardiography will be incorporated into upcoming models of pacemakers to allow for early detection of a falling intrathoracic impedance, which in and of itself is an indication of impending congestive heart failure. Significantly further benefit will accrue if the calculation of impedance can lead to an accurate calculation of cardiac output, which in turn will lead to more precise manipulation of medical and pacemaker therapies being applied to the management of congestive heart failure.

Therefore, there exists a need for a method to optimize cardiac resynchronization therapy using a pacemaker that uses impedance cardiography to calculate not only impedance, but cardiac output, accurately.

### BRIEF SUMMARY OF THE INVENTION

The foregoing needs are met by the present invention which provides an implantable pacemaker device with an algorithm capable of calculating cardiac output, in turn allowing for simple, accurate and real-time optimization of cardiac resynchronization therapy without the need for echocardiography. The implantable device is capable of accurate measurement of impedance, which can be interrogated transdermally in a clinic or outpatient setting, much as current pacemakers are interrogated today, by the treating physician (the cardiologist or electrophysiologist). The ability to obtain this data and convert it to cardiac output simply, accurately and cost effectively, would be expected to revolutionize the management of congestive heart failure and possibly impact not only the cost of the disease, but its morbidity and mortality.

Such an implantable device allows not only for optimization of cardiac resynchronization therapy in patients who fail to respond to therapy, but would allow for optimization of medical therapy, such as dose adjustment of medications, adding or subtracting therapies as cardiac output is positively or negatively impacted; adjusting CPAP or biPAP settings in patients being treated for sleep apnea; monitoring cardiac function easily and inexpensively in patients receiving chemotherapy (much of which is cardiotoxic); risk assessment in any or all post myocardial infarction patients, and so forth.

It is therefore an advantage of the present invention to provide a method for optimizing cardiac resynchronization therapy using a pacemaker that uses impedance cardiography to calculate cardiac output.

These and other features, aspects, and advantages of the present invention will become better understood upon consideration of the following detailed description, drawing, and appended claims.

### BRIEF DESCRIPTION OF THE DRAWING

FIG. 1 is a schematic of a cardiac pacing system used with the invention.

### DETAILED DESCRIPTION

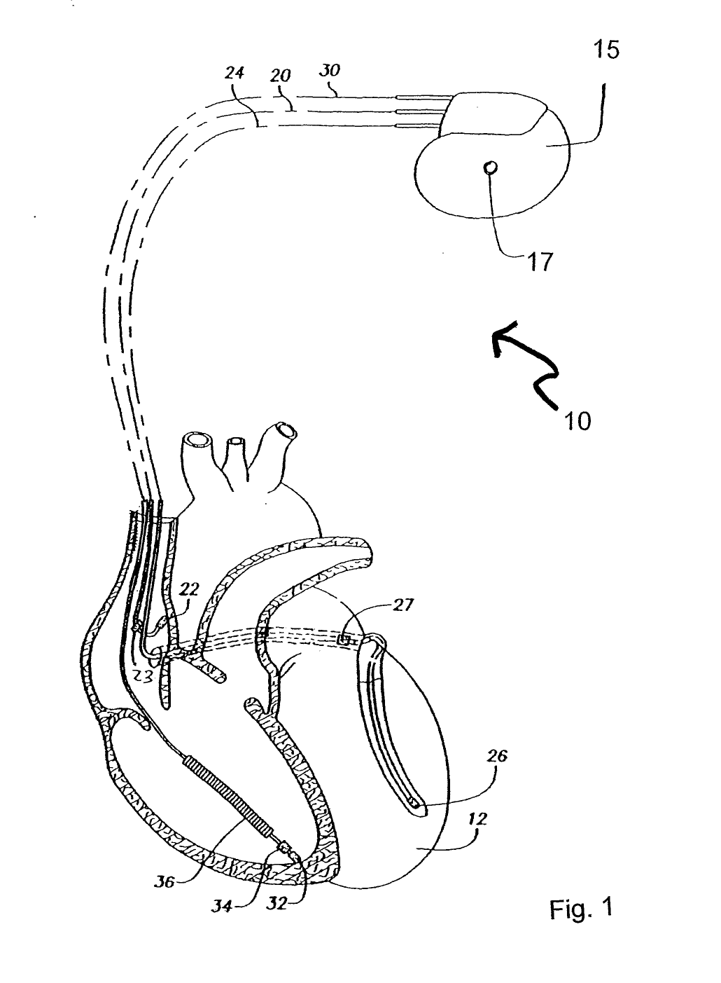

In FIG. 1, there is shown a cardiac pacing system 10 suitable for use with the present invention. The cardiac pacing system 10 includes a pacemaker 15 having a circuit in electrical communication with a patient's heart 12 by way of three leads 20, 24 and 30 suitable for delivering multi-chamber stimulation and shock therapy. The circuit of the pacemaker 15 is also in communication with an electrode 17 that is located on or near the pacemaker. The pacemaker 15 is implanted subcutaneously in the patient's body between the skin and upper ribs. The pacemaker 15 provides stimulating pulses from a pulse generator to the heart.

To sense right atrial cardiac signals and to provide right atrial chamber stimulation therapy, the pacemaker 15 is coupled to an implantable right atrial lead 20 having a right atrial tip electrode 22, which typically is implanted in the patient's right atrial appendage. The right atrial lead 20 may also have a right atrial ring electrode 23 to allow bipolar stimulation or sensing in combination with the right atrial tip electrode 22.

To sense left ventricular cardiac signals and to provide left-chamber stimulation therapy, the pacemaker 15 is coupled to a coronary sinus lead 24 designed for placement in the coronary sinus region via the coronary sinus ostium so as to place a distal electrode adjacent to the left ventricle. The coronary sinus lead 24 is designed to receive left ventricular cardiac signals and to deliver left ventricular stimulation therapy using a left ventricular tip electrode 26.

The pacemaker 15 is also shown in electrical communication with the patient's heart 12 by way of an implantable right ventricular lead 30 having a right ventricular tip electrode 32, a right ventricular ring electrode 34, and a right ventricular coil electrode 36. Typically, the right ventricular lead 30 is transvenously inserted into the heart 12 so as to place the right ventricular tip electrode 32 in the right ventricular apex so that the right ventricular coil electrode 36 will be positioned in the right ventricle. Accordingly, the right ventricular lead 30 is capable of receiving cardiac signals, and delivering stimulation in the form of pacing and shock therapy to the right ventricle.

During operation, the pacemaker 15 provides an alternating current signal between the pacemaker 15 and the left ventricular tip electrode 26. The electrode 17 on or near the pacemaker 15 and a coronary sinus ring electrode 27 (or left ventricular tip electrode 26, or right ventricular tip electrode 32, or right ventricular ring electrode 34, or right atrial tip electrode 22) provide signals representative of impedance changes between the pacemaker 15 and the heart to the circuit in the pacemaker 15. The circuit in the pacemaker 15 includes a microprocessor having software or firmware for storing the impedance data.

The pacemaker 15 also provides pacing pulses to the atrial tip electrode 22, the right ventricular tip electrode 32 and the left ventricular tip electrode 26 during operation. In order to maximize cardiac output, the pacing pulse interval between the atrial tip electrode 22 and the right ventricular tip electrode 32 must be optimized, and the pacing pulse interval between the right ventricular tip electrode 32 and the left ventricular tip electrode 26 must be optimized, along with other appropriate programmable parameters.

The circuit of the pacemaker 15 also includes a receiver capable of receiving interrogation signals (such as radio frequency signals) from an external computing device. The interrogation signals pass through the receiver to the control logic in the pacemaker microprocessor memory. The memory will produce information relating to interrogation signals and generate this data back through the control logic into a transmitter in the pacemaker so that the transmitter transmits this data to the external computing device. For example, the external computing device may send interrogation signals requesting the impedance values from the pacemaker 15. The interrogation signals pass through the receiver in the pacemaker 15 to the control logic and impedance data is generated back through the transmitter to the external computing device where cardiac output is calculated from the impedance data. As is known, a measure of cardiac output can be obtained by extracting the first time derivative of cyclical impedance changes. Suitable software, including a proprietary algorithm for calculating cardiac output from impedance, is available from Vasamed, Minneapolis, Minn., USA. The cardiac output value may be displayed on the display of the external computing device. In a similar manner, the external computing device may send interrogation signals requesting the current pacing pulse interval between the atrial tip electrode 22 and the right ventricular tip electrode 32, and the current pacing pulse interval between the right ventricular tip electrode 32 and the left ventricular tip electrode 26. This pacing pulse interval data may then be displayed on the display of the external computing device (e.g., a laptop computer).

Having described the components of a cardiac pacing system 10 suitable for use with the present invention, various methods of the invention can be described.

In one version of the invention, there is provided a method for optimizing cardiac resynchronization therapy using the cardiac pacing system 10. First, a timing interval between successive right atrial stimulation pulses, which are provided from the pacemaker pulse generator to the right atrial tip electrode 22, and right ventricular stimulation pulses, which are provided from the pacemaker pulse generator to the right ventricular tip electrode 32, is stored in a memory location in the pacemaker microprocessor. An alternating current signal is generated between the pacemaker 15 and the left ventricular tip electrode 26. Impedance changes are sensed between the electrode 17 on or near the pacemaker 15 and the coronary sinus ring electrode 27 (or left ventricular tip electrode 26, or right ventricular tip electrode 32, or right ventricular ring electrode 34, or right atrial tip electrode 22) to provide signals representative of impedance.

Then, impedance values are transmitted to a computing device external to the patient, thus allowing the calculation of cardiac output in the computing device and display of the calculated cardiac output values on a display of the computing device. The timing interval in the pacemaker microprocessor memory location can be adjusted by transmitting signals to the microprocessor from the computing device. Also, the cardiac output values as a function of time may be stored in the microprocessor or on the computing device for analysis.

In another version of the invention, there is provided a method for optimizing cardiac resynchronization therapy using the cardiac pacing system 10. First, a timing interval between successive right ventrical stimulation pulses, which are provided from the pacemaker pulse generator to the right ventricular tip electrode 32, and left ventricular stimulation pulses, which are provided from the pacemaker pulse generator to the left ventricular tip electrode 26, is stored in the pacemaker microprocessor. An alternating current signal is generated between the pacemaker 15 and the left ventricular tip electrode 26. Impedance changes are sensed between the electrode 17 on or near the pacemaker 15 and the coronary sinus ring electrode 27 (or left ventricular tip electrode 26, or right ventricular tip electrode 32, or right ventricular ring electrode 34, or right atrial tip electrode 22) to provide signals representative of impedance.

Then, impedance values are transmitted to a computing device external to the patient, thus allowing the calculation of cardiac output in the computing device and display of the calculated cardiac output values on a display of the computing device. The timing interval in the pacemaker microprocessor memory location can be adjusted by transmitting signals to the microprocessor from the computing device. Also, the cardiac output values as a function of time may be stored in the microprocessor or on the computing device for analysis.

In yet another version of the invention, there is provided a method for adjusting dosage of a medication in a patient having a pacemaker located external to the patient's heart. In the method, an alternating current signal is generated between the pacemaker 15 and the left ventricular tip electrode 26. Impedance changes are sensed between the electrode 17 on or near the pacemaker 15 and the coronary sinus ring electrode 27 (or left ventricular tip electrode 26, or right ventricular tip electrode 32, or right ventricular ring electrode 34, or right atrial tip electrode 22) to provide signals representative of impedance. Then, impedance signals are transmitted from the microprocessor to an external computing device, and cardiac output values are calculated from impedance signals received in the external computing device. The cardiac output values as a function of time are stored in the microprocessor or in the external computing device and then reviewed by a physician. The dosage of the medication may then be adjusted based on the stored cardiac output values.

Thus, the present invention provides an implantable pacemaker that uses impedance cardiography to calculate cardiac output, and in so doing, a method for optimizing cardiac resynchronization therapy by interrogating the CRT device, itself, or of a multitude of other therapies.

Although the present invention has been described with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

1. A method for optimizing cardiac resynchronization therapy in a cardiac pacing system including a pacemaker located external to a patient's heart, the pacemaker including a pulse generator and a microprocessor, the pacemaker providing right atrial stimulation via a right atrial electrode, the pacemaker providing ventricular stimulation to both right and left ventricles, the method comprising:
storing, in the microprocessor, timing intervals between successive right atrial stimulation pulses and right and left ventricular stimulation pulses;
generating an alternating current signal between the pacemaker and the heart;
sensing impedance between a current source on or near the pacemaker and a sensor in or on the heart;
calculating impedance between the current source and the sensor; and
calculating cardiac output to allow for the adjustment of the timing intervals in the microprocessor to maximize the efficacy of the cardiac resynchronization therapy.

2. The method of claim 1 further comprising:
transmitting the impedance values to a computing device external to the patient;
calculating cardiac output values in the computing device utilizing an algorithm, and
displaying the cardiac output values on a display of the computing device,
wherein the timing interval is adjusted by transmitting signals to the microprocessor from the computing device.

3. The method of claim 2 wherein:
the timing interval is adjusted based on the displayed the cardiac output values.

4. The method of claim 3 wherein:
the timing interval is adjusted to maximize cardiac output.

5. The method of claim 1 further comprising:
storing the cardiac output values as a function of time on the computing device for analysis.

6. The method of claim 1 wherein:
the current source is an electrode on the pacemaker.

7. The method of claim 1 wherein:
the sensor is the right atrial tip electrode, or
the sensor is a coronary sinus ring electrode, or
the sensor is a left ventricular tip electrode, or
the sensor is a right ventricular tip electrode, or
the sensor is a right ventricular ring electrode.

8. (canceled)

9. (canceled)

10. (canceled)

11. (canceled)

12. The method of claim 1 wherein:
cardiac output is calculated from impedance changes.

13. A method for optimizing cardiac resynchronization therapy in a cardiac pacing system including a pacemaker located external to a patient's heart, the pacemaker including a pulse generator and a microprocessor, the pacemaker providing right ventricular stimulation pulses via a right ventricle electrode in the right ventricle and the pacemaker, providing left ventricular stimulation pulses to a left ventricle electrode external to the left ventricle, the method comprising:
storing, in the microprocessor, a timing interval between successive right ventricular stimulation pulses and left ventricular stimulation pulses;
generating an alternating current signal between the pacemaker and the heart;
sensing impedance between a current source on or near the pacemaker and a sensor in or on the heart;
calculating impedance signals received in the microprocessor from the current source and the sensor; and
transmitting the calculated impedance signals to an analyzer external to the patient;
calculating real-time cardiac output values in the analyzer; and
reviewing the cardiac output values and adjusting the timing interval in the microprocessor to maximize the cardiac output.

14. The method of claim 13 further comprising:
displaying the cardiac output values on a display of the analyzer,
wherein the timing interval is adjusted by transmitting signals to the microprocessor from the analyzer.

15. The method of claim 13 further comprising:
storing the cardiac output values as a function of time on the analyzer for analysis.

16. The method of claim 13 wherein:
the current source is an electrode on the pacemaker.

17. The method of claim 13 wherein:
the sensor is the left ventricular electrode, or
the sensor is the right ventricular electrode.

18. (canceled)

19. The method of claim 13 wherein:
cardiac output is calculated from impedance changes.

20. A system for optimizing cardiac resynchronization therapy for a patient's heart, the system comprising:
a pacemaker located external to the heart, the pacemaker having a pulse generator and a microprocessor;
a computing device in communication with the microprocessor, the computing device being external to the patient;
means for generating an alternating current signal between the pacemaker and the heart;
a current source on or near the pacemaker and a current sensor in or on the heart, the current source and the current sensor sensing impedance between the current source and the current sensor; the current source and the current sensor being in electrical communication with the microprocessor for providing impedance signals to the microprocessor;
a right atrium electrode in electrical communication with the pulse generator, the right atrium electrode delivering atrial stimulation pulses to the right atrium of the heart;
right and left ventricular electrodes in electrical communication with the pulse generator, the right ventricular electrode delivering ventricular stimulation pulses to the right ventricle of the heart, the left ventricular electrode delivering stimulation to the left ventricle,
wherein the microprocessor executes a stored program to:
(i) store inputted timing intervals between successive atrial stimulation pulses and ventricular stimulation pulses,
(ii) calculate intrathoracic impedance data, and
(iii) transmit the impedance data to the computing device.

21. The system of claim 20 wherein:
the computing device executes a stored program to convert the impedance data to cardiac output data and display the cardiac output data.

22. The system of claim 20 wherein:
the current source is an electrode on the pacemaker.

23. The system of claim 20 wherein:
the sensor is the left ventricular electrode, or
the sensor is the right ventricular electrode, or
the sensor is the right atrium electrode.

24. (canceled)

25. (canceled)

26. (canceled)

27. A system for optimizing cardiac resynchronization therapy for a patient's heart, the system comprising:
a pacemaker located external to the heart, the pacemaker having a pulse generator and a microprocessor;
a computing device in communication with the microprocessor, the computing device being external to the patient;
means for generating an alternating current signal between the pacemaker and the heart;
a current source on or near the pacemaker and a current sensor in or on the heart, the current source and the current sensor measuring impedance between the current source and the current sensor, the current source and the current sensor being in electrical communication with the microprocessor for providing impedance signals to the microprocessor;
a right ventricular electrode in electrical communication with the pulse generator, the right ventricular electrode delivering right ventricular stimulation pulses to the right ventricle of the heart; and
a left ventricular electrode in electrical communication with the pulse generator, the left ventricular electrode delivering left ventricular stimulation pulses to the left ventricle of the heart,
wherein the microprocessor executes a stored program to:
(i) store inputted timing intervals between successive right ventricular stimulation pulses and left ventricular stimulation pulses,
(ii) calculate impedance values, and
(iii) transmit the timing intervals and the impedance values to the computing device, allowing for calculation of cardiac output and optimization of the cardiac resynchronization therapy system.

28. The system of claim 27 wherein:
the current source is an electrode on the pacemaker.

29. The system of claim 27 wherein:
the sensor is the left ventricular electrode or
the sensor is the right ventricular electrode.

30. (canceled)

31. (canceled)

32. The system of claim 27 wherein:
the computing device executes a stored program to convert the impedance data to cardiac output data and display the cardiac output data.

33. A method for adjusting dosage of a medication in a patient having a pacemaker located external to the patient's heart, the pacemaker including a microprocessor, the method comprising:
generating an alternating current signal between the pacemaker and the heart;
sensing impedance between a current source on or near the pacemaker and a current sensor in or on the heart;
calculating, in the microprocessor, impedance data between the current source and the current sensor;
transmitting the impedance data to an external PC based analyzer;
calculating cardiac output values in the analyzer from the impedance data;
reviewing the cardiac output values; and
adjusting dosage of the medication based on the cardiac output values.

34. The method of claim 33 wherein:
the current source is an electrode on the pacemaker.

35. The method of claim 33 wherein:
the sensor is a right atrial tip electrode, or
the sensor is a coronary sinus ring electrode, or
the sensor is a left ventricular tip electrode, or
the sensor is a right ventricular tip electrode, or
the sensor is a right ventricular ring electrode.

36. (canceled)

37. (canceled)

38. (canceled)

39. (canceled)
